(19) United States
(12) Patent Application Publication
(10) Pub. No.: US 2019/0262494 A1
(43) Pub. Date: Aug. 29, 2019

(54) TOPICAL DRESSING COMPOSITION FOR THE TREATMENT OF DAMAGED SKIN TISSUE

(71) Applicant: Habil F KHORAKIWALA, Aurangabad, Maharashtra (IN)

(72) Inventors: Habil F KHORAKIWALA, Aurangabad (IN); Zahabiya KHORAKIWALA, Aurangabad (IN); Vijay SHARMA, Aurangabad (IN)

(21) Appl. No.: 16/342,850

(22) PCT Filed: Oct. 17, 2017
(86) PCT No.: PCT/IN2017/050478

§ 371 Date: Apr. 17, 2019

(30) Foreign Application Priority Data
Oct. 19, 2016 (IN) 201621035700
Oct. 19, 2016 (IN) 201621035701
Oct. 19, 2016 (IN) 201621035702
Oct. 19, 2016 (IN) 201621035703

(51) Int. Cl. A61L 15/40; A61L 15/28; A61L 26/00; A61F 13/00; A61M 35/00; B29C 39/00; B29C 39/10; B29C 39/38

(52) U.S. Cl. CPC A61L 15/40; B29L 2031/753; A61L 26/0057; A61L 26/0023; A61F 13/00063; A61M 35/00; A61F 13/00991; B29C 39/003; B29C 39/10; B29C 39/38; A61L 2430/34; A61L 2300/64; A61L 2300/412; B29K 2005/00; B29K 2905/12; A61L 15/28

(57) ABSTRACT

The present invention relates to a topical dressing composition for the treatment of damaged skin tissue in a subject, wherein said topical dressing comprises mesenchymal stem cells embedded in a topical matrix. Particularly the invention relates to a storage-stable topical dressing composition for the treatment of damaged skin tissue in a subject, wherein said topical dressing comprises up to about 40,000 mesenchymal stem cells per square centimetre embedded in a topical matrix and, optionally a pharmaceutically acceptable excipient.

### TECHNICAL FIELD OF THE INVENTION

The present invention relates to a topical dressing composition for the treatment of damaged skin tissue in a subject, wherein said topical dressing comprises mesenchymal stem cells embedded in a topical matrix. Particularly, the invention relates to a storage-stable topical dressing composition for the treatment of damaged skin tissue in a subject, wherein said topical dressing comprises up to about 40,000 mesenchymal stem cells per square centimetre embedded in a topical matrix, and optionally a pharmaceutically acceptable excipient.

### BACKGROUND OF THE INVENTION

Stem cells are characterized by their self-renewal ability and differentiation potential. These cells can be divided into embryonic and adult stem cells. Most adult stem cells are minor populations found in adult organs that can differentiate into specific cell types of their tissue of origin, e.g., mesenchymal stem cells. Several lines of evidence have shown that under appropriate environments, mesenchymal stem cells are able to differentiate into mesodermal, endodermal and even ectodermal cells. In addition, mesenchymal stem cells have the ability to migrate and engraft into host tissues that can help in repair and enhancement of tissue regeneration.

Mesenchymal stem cell-mediated immunoregulatory effects can be applied towards the management of various autoimmune disorders such as Crohn's disease, type 1 diabetes, multiple sclerosis, systemic lupus erythematosus, sjogren syndrome and systemic sclerosis. However, despite their profound effect on immune responses, these therapies do not induce clinically significant remissions in certain patients. Several literature articles and patents disclose the use of blood components isolated from patient for treatment of several diseases including autoimmune and skin disorders.

Mesenchymal stem cells are multipotent cells that can be found in several tissues such as bone marrow, adipose tissue, synovium, deciduous teeth, umbilical cord blood and blood vessels. Mesenchymal stem cells are a promising cell type for therapy, because they have the potential to differentiate into repair tissue and also have trophic and immunomodulatory capacities. While they have been shown to be capable of improving damaged tissue, their contribution does not seem to originate from long-term engraftment and differentiation. This suggests that mesenchymal stem cells can also stimulate endogenous tissue repair, in addition to their ability to differentiate into cells of the mesoderm lineage. Further, growth factors such as transforming growth factor β1 (TGF-β1) and vascular endothelial growth factor (VEGF), secreted by MSCs, have been shown to influence tissue repair and immunological processes. Mesenchymal stem cells also possess “anti-inflammatory” properties. These cells secrete factors that mitigate inflammation and promote wound healing of normal wounds.

Several literature articles and patents disclose the use of mesenchymal stem cells in treating skin diseases. Song et. al. (2016) discloses wound dressing of mesenchymal stem cells. U.S. Pat. No. 8,435,787 discloses microencapsulation of hepatocytes using alginate technology. Busscheet. al. (2015) discloses microencapsulated equine mesenchymal stromal cells for cutaneous wound healing. Schmitt et. al. (2015) discloses matrix of system containing human mesenchymal cells. Isakson et. al. (2015) discloses use of mesenchymal stem cells in cutaneous wound healing using a fibrin spray. Maxsonet. al. (2012) and Duscheret. al. (2014) disclose role of the mesenchymal stem cells in wound healing. US Publication No. 2012/0141433 discloses compositions of vaporized stem cell derivatives and methods for their use in the treatment of vascular disorders of the skin such as varicose veins, chronic (long-term) venous insufficiency, thrombophlebitis, and arteriovenous fistula.

Mesenchymal stem cells are known to be unstable and their viability is compromised when stored at ambient conditions. There is a need to develop storage-stable topical dressing compositions comprising mesenchymal stem cells, wherein most of the mesenchymal stem cells remain viable for the treatment of damaged skin tissue.

### SUMMARY OF THE INVENTION

In one general aspect of the invention, there is provided a topical dressing composition for the treatment of damaged skin tissue in a subject, wherein said topical dressing comprises mesenchymal stem cells embedded in topical matrix and optionally, a pharmaceutically acceptable excipient.

In another general aspect of the invention, there is provided a storage-stable topical dressing composition for the treatment of damaged skin tissue in a subject, wherein said topical dressing comprises up to about 40,000 mesenchymal stem cells per square centimetre embedded in a topical matrix and, optionally a pharmaceutically acceptable excipient, wherein at least about 80% of the contained mesenchymal stem cells remain viable when the composition is stored at about 25° C. for a period of at least 6 months.

In another general aspect of the invention, there is provided a storage-stable topical dressing composition in the form of a bandage or patch for the treatment of damaged skin tissue in a subject, wherein said bandage or patch comprises up to about 40,000 mesenchymal stem cells per square centimetre embedded in an topical matrix and, optionally a pharmaceutically acceptable excipient, wherein at least about 80% of the mesenchymal stem cells remain viable when the composition is stored at about 25° C. for a period of at least 6 months.

In another general aspect of the invention, there is provided a storage-stable topical dressing composition in the form of a dual chambered spray for the treatment of damaged skin tissue in a subject, said composition comprises (i) mesenchymal stem cells and topical matrix component in one chamber, and (ii) gelling component in another chamber, wherein more than about 80% of the mesenchymal stem cells remain viable when the composition is stored at about 25° C. for a period of at least 6 months.

In another embodiment, the mesenchymal stem cells component (one chamber) is separated from the gelling component (another chamber) that comprises calcium chloride solution.

In another general aspect of the invention, there is provided a storage-stable topical dressing composition in the form of a gel for the treatment of damaged skin tissue in a subject, wherein said gel comprises mesenchymal stem cells embedded in a topical matrix and, optionally a pharmaceutically acceptable excipient, wherein more than about 80% of the mesenchymal stem cells remain viable when the topical gel is stored at about 25° C. for a period of at least 6 months.

In another general aspect of the invention, there is provided a method of treatment of damaged skin tissue in a subject, said method comprising: (i) applying to the damaged skin tissue area a composition containing up to about 40,000 mesenchymal stem cells per square centimetre and a pharmaceutically acceptable excipient, and (ii) applying a topical matrix in the form of a lyophilized powder and optionally a pharmaceutically acceptable excipient onto the mesenchymal stem cell composition, thereby forming an in situ sponge on the damaged skin tissue.

In an embodiment of the present invention, the damaged skin tissue comprises traumatic wound, surgical wound, diabetic ulcer, pressure ulcer, venous ulcer, a scar, burn, a skin lesion, eczema, and a skin ulcer. Non-limiting examples of the damaged skin tissue include diabetic foot ulcer, pemphigus vulgaris, and epidermolysisbullosa, impetigo, hidradenitis suppurativa, keloids, lichen planus.

In another embodiment of the present invention, the mesenchymal stem cells are derived from adipose tissue, bone marrow, Whartons jelly dental tissue or umbilical cord of a subject, preferably the mesenchymal stem cells are derived from adipose tissue; and wherein the mesenchymal stem cells are suitable for autologous transfer or allogeneic transfer.

In another embodiment of the present invention, the topical dressing composition comprises growth factors selected from vascular endothelial growth factor, hepatocyte growth factor, fibroblast growth factor, and epidermal growth factor.

In an embodiment of the present invention, the topical dressing comprises up to about 40,000, or about 35,000, or about 30,000, or about 25,000, or about 20,000, or about 15,000 or about 10,000 mesenchymal stem cells per square centimetre. Preferably, the topical dressing contains about 10,000 to about 30,000, or about 15,000 to about 25,000, or about 20,000 mesenchymal stem cells per square centimetre.

In another embodiment of the present invention, more than about 85%, or more than about 80% of the contained mesenchymal stem cells remain viable when the composition is stored at about 25° C. for a period of at least about 6 months. Preferably, the mesenchymal stem cells remain viable when the composition is stored at about 25° C. for a period of about 12 months, or about 18 months, or about 24 months.

In an embodiment, the composition is also stable when stored at about 2° C. to about 8° C. Preferably, the mesenchymal stem cells remain viable when the composition is stored at about 2° C. to about 8° C. for a period of about 6 months, about 12 months, or about 18 months, or about 24 months.

In yet another embodiment of the present invention the topical matrix comprises alginate, polyurethane, collagen, chitosan, pectin, and hyaluronic acid. In another embodiment, the topical matrix comprises 2% w/v of alginate solution, or 2% w/v of chitosan solution, or 2% w/v of hyaluronic acid solution.

In another embodiment of the present invention the composition is in the form of a solution, suspension, emulsion, ointment, foam, paste, gel, spray, bandage, patch, cream, lotion or powder. In the context of the present invention, the composition is in the form of a bandage or patch.

In another embodiment of the present invention, the bandage or patch is sterile, wherein the bandage or patch has thickness in the range of about 0.5 mm to about 10 mm.

In another embodiment of the present invention, there is provided a method of preparation of a storage-stable topical dressing composition in the form of a bandage or patch wherein said method comprises the steps of:
a) placing sterile gauze sheet on to stainless steel casting mould;
b) applying mesenchymal stem cells and alginate matrix on the sterile gauze sheet;
c) placing casting mould in a bath of calcium chloride solution;
d) transferring of the casting mould to a bath containing phosphate buffer solution to wash excess of calcium;
e) placing of gauze sheet to a culture media flask for incubation;
f) incubating the gauze sheet at 37° C. at 5% CO₂ for 14 days; and
g) placing the incubated gauze sheet in trilaminated pouch to obtain the topical dressing composition.

In another embodiment, the present invention relates to a method of stabilizing mesenchymal stem cells in a topical dressing composition, said method comprising embedding up to about 40,000 mesenchymal stem cells per square centimetre in a topical matrix along with a pharmaceutically acceptable excipient, wherein at least about 80% of the contained mesenchymal stem cells remain viable when the composition is stored at about 25° C. for a period of at least about 6 months.

### BRIEF DESCRIPTION OF THE DRAWINGS

FIG. 1 shows photographic comparison of wound surface before and after application of topical dressing composition for 6 weeks in a human subject.

FIG. 2 shows photographic comparison of wound surface before and after application of topical dressing composition for 3 months in a human subject.

FIG. 3 shows photographic presentation of mesenchymal stem cells impregnated onto the sponge in a human subject.

### DETAILED DESCRIPTION OF THE INVENTION

While the invention has been described in term of its specific embodiments, certain modification and equivalents will be apparent to those skilled in the art and are intended to be included within the scope of the invention.

The inventors of the present invention have surprisingly found the beneficial use of mesenchymal stem cells for the treatment of damaged skin tissue in a subject such as acute or chronic wound. The inventors have invented a stable and effective topical dressing composition for repairing damaged skin tissue, said composition comprising mesenchymal stem cells embedded in a topical matrix and, optionally a pharmaceutically acceptable excipient.

In one general aspect of the invention there is provided, a topical dressing composition for the treatment of damaged skin tissue in a subject, wherein said topical dressing comprises mesenchymal stem cells embedded in a topical matrix and optionally, a pharmaceutically acceptable excipient.

In another aspect of the invention, there is provided a storage-stable topical dressing composition for the treatment of damaged skin tissue in a subject, wherein said topical dressing comprises up to about 40,000 mesenchymal stem cells per square centimetre embedded in a topical matrix and, optionally a pharmaceutically acceptable excipient, wherein at least about 80% of the contained mesenchymal stem cells remain viable when the composition is stored at about 25° C. for a period of at least 6 months.

In another aspect of the invention, there is provided a storage-stable topical dressing composition in the form of a bandage or patch for the treatment of damaged skin tissue in a subject, wherein said bandage or patch comprises up to about 40,000 mesenchymal stem cells per square centimetre embedded in an topical matrix and, optionally a pharmaceutically acceptable excipient, wherein at least about 80% of the mesenchymal stem cells remain viable when the composition is stored at about 25° C. for a period of at least 6 months.

In another aspect of the invention, there is provided a storage-stable topical dressing composition in the form of a dual chambered spray for the treatment of damaged skin tissue in a subject, said composition comprises (i) mesenchymal stem cells and topical matrix component in one chamber, and (ii) gelling component in another chamber, wherein more than about 80% of the mesenchymal stem cells remain viable when the composition is stored at about 25° C. for a period of at least 6 months.

In another embodiment, the gelling component comprises calcium chloride solution contained in the second chamber which is physically separated from the first chamber. In another embodiment the calcium chloride solution does not mix with mesenchymal stem cell component in the spray upon actuation.

In another general aspect of the invention, there is provided a storage-stable topical dressing composition in the form of a gel for the treatment of damaged skin tissue in a subject, wherein said gel comprises mesenchymal stem cells embedded in a topical matrix and, optionally a pharmaceutically acceptable excipient, wherein more than about 80% of the mesenchymal stem cells remain viable when the topical gel is stored at about 25° C. for a period of at least 6 months.

In another general aspect of the invention, there is provided a method of treatment of damaged skin tissue in a subject, said method comprising: (i) applying to the damaged skin tissue area a composition containing up to about 40,000 mesenchymal stem cells per square centimetre and a pharmaceutically acceptable excipient, and (ii) applying a topical matrix in the form of a lyophilized powder and optionally a pharmaceutically acceptable excipient onto the mesenchymal stem cell composition, thereby forming an in situ sponge on the damaged skin tissue.

As used herein, the term “storage stable” relates to a topical dressing composition comprising mesenchymal stem cells, wherein at least about 80% of the mesenchymal stem cells remain viable when the composition is stored at about 25° C. for a period of at least 6 months.

As used herein, the term “topical dressing” relates to the external application of the invention composition at the site of damages skin tissue. Accordingly, such topical dressing compositions are useful in the invention includes those pharmaceutical forms in which the composition is applied externally by direct contact with the skin surface to be treated. As used herein, the term “treatment” refers to beneficial or desired clinical results from the topical dressing composition of the invention.

As used herein, the term “damaged skin tissue” refers to skin tissue having any type of damage.

As used herein, the term “viable” refers to the mesenchymal stem cells that remain active and functional.

As used herein, the term “mesenchymal stem cells” refers to mesenchymal stem cells derived from adipose tissue, bone marrow, Whartons jelly, dental tissue or umbilical cord of a subject.

As used herein, the term “autologous” means cells or tissues derived from the same subject. As used herein, the term “allogeneic” means cells or tissues derived from another, genetically dissimilar subject of the same species.

In an embodiment of the present invention, the damaged skin tissue includes an acute wound and/or a chronic wound.

Mesenchymal stem cells are non-hematopoietic, multipotent cells that can differentiate into a variety of different cell types and give rise to bones, cartilage and other mesenchymal tissues. Mesenchymal stem cells are characterized morphologically by a small cell body with a few cell processes. The cell body contains a large, round nucleus with a prominent nucleolus, which is surrounded by finely dispersed chromatin particles, giving the nucleus a clear appearance. The remainder of the cell body contains a small amount of Golgi apparatus, rough endoplasmic reticulum, mitochondria and polyribosomes. The cells, which are long and thin, are widely dispersed and the adjacent extracellular matrix is populated by a few reticular fibrils but is devoid of the other types of collagen fibrils.

In an embodiment of the present invention, the mesenchymal stem cells are derived from adipose tissue, bone marrow, Whartons jelly dental tissue or umbilical cord of a subject. Preferably the mesenchymal stem cells are derived from adipose tissue and such mesenchymal stem cells are suitable for autologous transfer or allogeneic transfer.

In another embodiment of the present invention, the composition comprises growth factors selected from vascular endothelial growth factor, hepatocyte growth factor, fibroblast growth factor, and epidermal growth factor.

In an embodiment of the present invention, the topical dressing comprises about 10,000 to about 30,000, or about 15,000 to about 25,000, or about 20,000 mesenchymal stem cells per square centimetre.

In another embodiment, the topical dressing composition comprises about 10,000, or about 15,000, or about 20,000, or about 25,000, or about 30,000, or about 35,000, or about 40,000 mesenchymal stem cells per square centimetre. Each amount constitutes an alternate embodiment of the present invention.

In another embodiment of the present invention, more than about 85%, or more than about 80% of the contained mesenchymal stem cells remain viable when the composition is stored at about 25° C. for a period of at least about 6 months. Preferably, the mesenchymal stem cells remain viable when the composition is stored at about 25° C. for a period of about 12 months, or about 18 months, or about 24 months.

In an embodiment, the composition is also stable when stored at about 2° C. to about 8° C. Preferably, the mesenchymal stem cells remain viable when the composition is stored at about 2° C. to about 8° C. for a period of about 6 months, about 12 months, or about 18 months, or about 24 months.

Topical matrix is a potential solution to decrease mesenchymal stem cell's tendency to migrate from the wound site. Matrix helps to immobilize the cells in order to the cell survival. In another embodiment of the present invention, the topical matrix comprises alginate, polyurethane, collagen, chitosan, pectin, and hyaluronic acid. In another embodiment the topical matrix comprises 2% w/v of alginate solution, or 2% w/v of chitosan solution, or 2% w/v of hyaluronic acid solution.

In another embodiment of the present invention, the composition is in the form of a solution, suspension, emulsion, ointment, foam, paste, gel, spray, bandage, patch, cream, lotion or powder. In the context of the present invention, the composition is in the form of a bandage or patch.

In another embodiment of the present invention, the bandage or patch is sterile, and wherein the bandage or patch has thickness in the range of about 0.5 mm to about 10 mm, or about 1 mm, or about 2 mm, or about 3 mm, or about 4 mm, or about 5 mm, or about 6 mm, or about 7 mm, or about 8 mm, or about 9 mm. Each amount constitutes an alternate embodiment of the present invention.

In another embodiment of the present invention, there is provided a method of preparation of a storage-stable topical dressing composition in the form of a bandage or patch wherein said method comprises the steps of:
a) placing sterile gauze sheet on to stainless steel casting mould;
b) applying mesenchymal stem cells and alginate matrix on the sterile gauze sheet;
c) placing the casting mould in a bath of calcium chloride solution;
d) transferring of the casting mould to a bath containing phosphate buffer solution to wash excess of calcium;
e) placing of gauze sheet to a culture media flask for incubation;
f) incubating the gauze sheet at 37° C. at 5% CO₂ for 14 days; and
g) placing the incubated gauze sheet in trilaminated pouch to obtain the topical dressing composition.

In an embodiment of the invention, the topical dressing composition comprises optionally a pharmaceutically acceptable excipients selected from thickening agent, buffer, surfactant, antioxidant, stabilizer and solvent.

A “thickening agent” as used herein include, but not limited to one or more of anionic cellulose materials, such as sodium carboxy methyl cellulose; anionic polymers such as carboxy vinyl polymers; nonionic cellulose materials, such a methyl cellulose and hydroxy propyl methyl cellulose; hydroxy ethyl cellulose; cationic cellulose materials, such as Polymer JR 400; cationic gum materials, such as Jaguar C₁₃S; other gum materials such as gum acacia, gum tragacanth, locust bean gum, guar gum and carrageenan; proteins, such as albumin and protein hydrolysates; and clay materials, such as bentonite, hectorite, magnesium aluminium silicate, sodium magnesium silicate and combination thereof. Preferred thickening agent is hydroxy ethyl cellulose.

The concentration of thickening agent ranges from about 5% to about 25% by weight of the composition, or, about 10%, or about 15%, or about 20% by weight of the composition. Each of this concentration constitutes an alternate embodiment of the invention. Preferred concentration is about 5%, or about 10%, or about 15%, or about 20% by weight of the composition.

The “preservatives” as used herein include, but are not limited to one or more of ethanol, benzoic acid, sodium benzoate, sorbic acid, potassium sorbate, sodium propionate and the methyl, ethyl, propyl and butyl esters of p-hydroxybenzoic acid 2-bromo-2-nitropropane-1,3-diol, phenoxyethanol, dibromodicyanobutane, formalin, triclosan and combination thereof. The concentration of preservative ranges from 0.1% to 2% by weight of the composition, or about 0.1%, or about 0.2%, or about 0.3%, or about 0.4%, or about 0.5%, or about 0.6%, or about 0.7%, or about 0.8%, or about 0.9%, or about 1.0%, or about 1.1%, or about 1.2%, or about 1.3%, or about 1.4%, or about 1.5%, or about 1.6%, or about 1.7%, or about 1.8%, or about 1.9%, or about 2% by weight of the composition. Preferred concentration of the preservative is about 0.3%, or about 0.45%, or about 0.5%, or about 0.65%, or about 0.75% by weight of the composition.

The “buffering agent” as used herein include, but are not limited to one or more of citric acid, citric acid monohydrate, boric acid, and phosphoric acid, sodium citrate, sodium citrate dihydrate, monopotassium phosphate, disodium phosphate and combination thereof.

The “surfactant” as used herein can be selected from the group comprising of one or more of sodium bis(2-ethylhexyl)sulfosuccinate, sodium bis(tridecyl)sulfosuccinate, bis(dialkyl)sulfosuccinate salts, copolymers of polydimethylsiloxane and polyethylene/polypropylene-oxide, polyoxypropylene (12) dimethicone, cetyl PEG/PPG-10/1 dimethicone, hexyl laurate and polyglyceryl-4-isostearate, PEG-10 dimethicone, sorbitanmonolaurate, sorbitanmonooleate, polyoxyethylene (20) sorbitanmonooleate (Polysorbate 80), polyethoxylated castor oil, polyoxyethylenesorbitantrioleate, polyoxyethyleneoctyl phenyl ether, polyoxyethylene 20 cetyl ether, polyethylene glycol tert-octylphenyl ether, sodium di(2-ethylhexyl)phosphate, sodium di(oleyl)phosphate, sodium di(tridecyl)phosphate, sodium dodecylbenzenesulfonate, sodium 3-dodecylaminopropanesulfonate, sodium 3-dodecylaminopropionate, sodium N-2-hydroxydodecyl-N-methyltaurate, lecithin, sucrose fatty acid esters, 2-ethylhexylglycerin, caprylyl glycol, long chain hydrophobic vicinal diols of monoalkyl glycols, monoalkylglycerols, or monoacylglycerols, polyoxyl castor oil derivatives, polyethylene glycol hydrogenated castor oil, potassium oleate, sodium oleate, cetylpyridynium chloride, alkyltrimethylammonium bromides, benzalkonium chloride, didodecyldimethylammonium bromide, trioctylmethylammonium bromide, cetyltrimethylammonium bromide, cetyldimethylethylammonium bromide, and combinations thereof. The preferred surfactants are polysorbate 80, sodium oleate, lecithin, sucrose fatty acid esters, and polyoxyl castor oil derivatives. Each constitutes an alternate embodiment of the invention.

The suitable “antioxidant” as used herein can be selected from the group comprising of one or more of acetyl cysteine, ascorbic acid, ascorbic acid polypeptide, ascorbyldipalmitate, ascorbylmethylsilanolpectinate, ascorbylpalmitate, ascorbyl stearate, BHA, BHT, t-butyl hydroquinone, cysteine, cysteine HCl, diamylhydroquinone, di-t-butylhydroquinone, dicetylthiodipropionate, dioleyltocopherylmethylsilanol, disodium ascorbylsulfate, distearylthiodipropionate, ditridecylthiodipropionate, dodecyl gallate, erythorbic acid, esters of ascorbic acid, ethyl ferulate, ferulic acid, gallic acid esters, hydroquinone, isooctylthioglycolate, kojic acid, magnesium ascorbate, magnesium ascorbyl phosphate, methylsilanolascorbate, natural botanical anti-oxidants such as green tea or grape seed extracts, nordihydroguaiaretic acid, octylgallate, phenylthioglycolic acid, potassium ascorbyltocopheryl phosphate, potassium sulfite, propyl gallate, quinones, rosmarinic acid, sodium ascorbate, sodium bisulfite, sodium erythorbate, sodium metabisulfite, sodium sulfite, superoxide dismutase, sodium thioglycolate, sorbityl furfural, thiodiglycol, thiodiglycolamide, thiodiglycolic acid, thioglycolic acid, thiolactic acid, thiosalicylic acid, tocophereth-5, tocophereth-10, tocophereth-12, tocophereth-18, tocophereth-50, tocopherol, tocophersolan, tocopheryl acetate, tocopheryllinoleate, tocopherylnicotinate, tocopheryl succinate, tris(nonylphenyl)phosphate, and combination thereof.

In another embodiment of the present invention, themesenchymal stem cells are evaluated for their ability to differentiate using adipogenic, chondrogenic, and osteogenic differentiation media. The cell surface markers are evaluated by Flow cytometry. The viability of cells is assayed by dye exclusion 7AADanalysis on the flow cytometer. Cell cycle analysis and DNA ploidy is established using the Cycle Test BD and FITC BrdU Flow Kit (BD BisSciences). Mycoplasma and expression of sox2, nanog are done by PCR and RT-PCR respectively. Endotoxin and sterility is established using LAL assay kit and microbiological culture methods.

In another embodiment, the mesenchymal stem cells are grown and its suspension is prepared in growth medium known to person skilled in the art. The preferred media are selected from, but not limited to, phosphate buffer saline, normal saline, Dulbecco's modified Eagle's medium (DMEM), Hank's balanced salt solution (HBSS), DMEM-F12 and DMEM-low glucose. The most preferred medium is DMEM.

In another embodiment, the present invention relates to a method of stabilizing mesenchymal stem cells in a topical dressing composition, said method comprising embedding up to about 40,000 mesenchymal stem cells per square centimetre in a topical matrix along with a pharmaceutically acceptable excipient, wherein at least about 80% of the contained mesenchymal stem cells remain viable when the composition is stored at about 25° C. for a period of at least about 6 months.

In one embodiment of the present invention there is provided, a storage-stable topical dressing composition for the treatment of damaged skin tissue in a subject, wherein said topical dressing comprises about 10,000 to about 30,000 mesenchymal stem cells per square centimetre embedded in a topical matrix and, optionally a pharmaceutically acceptable excipient, wherein at least about 80% of the contained mesenchymal stem cells remain viable when the composition is stored at about 25° C. for a period of at least 6 months, or at least 12 months.

In one embodiment of the present invention there is provided, a storage-stable topical dressing composition for the treatment of damaged skin tissue in a subject, wherein said topical dressing comprises about 15,000 to about 25,000 mesenchymal stem cells per square centimetre embedded in a alginate matrix and, optionally a pharmaceutically acceptable excipient, wherein at least about 90% of the contained mesenchymal stem cells remain viable when the composition is stored at about 25° C. for a period of at least 6 months, or at least 12 months.

In one embodiment of the present invention there is provided, a storage-stable topical dressing composition for the treatment of damaged skin tissue in a subject, wherein said topical dressing comprises about 15,000 to about 22,000 mesenchymal stem cells per square centimetre embedded in a alginate matrix and, optionally a pharmaceutically acceptable excipient, wherein at least about 90% of the contained mesenchymal stem cells remain viable when the composition is stored at about 2° C. to about 8° C. for a period of at least 12 months, or at least 18 months.

In one embodiment of the present invention there is provided, a storage-stable topical dressing composition in the form of a bandage or patch for the treatment of damaged skin tissue in a subject, wherein said bandage or patch comprises about 18,000 to about 22,000 mesenchymal stem cells per square centimetre embedded in an alginate matrix and, optionally a pharmaceutically acceptable excipient, wherein at least about 90% of the mesenchymal stem cells remain viable when the composition is stored at about 25° C. for a period of at least 6 months, or at least 12 months.

In one embodiment of the present invention there is provided, a storage-stable topical dressing composition in the form of a bandage or patch for the treatment of damaged skin tissue in a subject, wherein said bandage or patch comprises about 20,000 mesenchymal stem cells per square centimetre embedded in an alginate matrix and, optionally a pharmaceutically acceptable excipient, wherein at least about 90% of the mesenchymal stem cells remain viable when the composition is stored at about 2° C. to about 8° C. for a period of at least 12 months, or at least 18 months.

In one embodiment of the present invention there is provided, a storage-stable topical dressing composition in the form of a sterile bandage or patch for the treatment of an acute and/or chronic wound in a subject, wherein said bandage or patch comprises about 18,000 to about 22,000 mesenchymal stem cells per square centimetre embedded in an alginate matrix and, a sterile gauze packed in a laminated pouch, wherein at least about 90% of the mesenchymal stem cells remain viable when the composition is stored at about 25° C. for a period of at least 6 months, or at least 12 months.

In one embodiment of the present invention there is provided, a pouch or sachet containing a topical dressing composition in the form of a sterile bandage or patch for the treatment of an acute and/or chronic wound in a subject, wherein said bandage or patch comprises about 18,000 to about 22,000 mesenchymal stem cells per square centimetre embedded in an alginate matrix and, a sterile gauze, wherein at least about 90% of the mesenchymal stem cells remain viable when the pouch or sachet is stored at about 25° C. for a period of at least 6 months, or at least 12 months. In an embodiment, the pouch or sachet is made up of a material comprising polyethylene terephthalate, polypropylene, aluminium, polyolefins, polyamide, polyvinyl chloride, ethyl vinylidine copolymer, and polystyrene.

In one embodiment of the present invention there is provided, a pouch or sachet containing a topical dressing composition in the form of a sterile bandage or patch for the treatment of an acute and/or chronic wound in a subject, wherein said bandage or patch comprises about 18,000 to about 22,000 mesenchymal stem cells per square centimetre embedded in an alginate matrix and, a sterile gauze, wherein at least about 90% of the mesenchymal stem cells remain viable when the pouch or sachet is stored at about 25° C. for a period of at least 6 months, or at least 12 months. In an embodiment, the pouch or sachet is made up of a material comprising polyethylene terephthalate, polypropylene, aluminium, polyolefins, polyamide, polyvinyl chloride, ethyl vinylidine copolymer, and polystyrene.

In one embodiment of the present invention there is provided, a kit containing (i) a topical dressing composition in the form of a sterile bandage or patch for the treatment of an acute and/or chronic wound in a subject, wherein said bandage or patch comprises about 18,000 to about 22,000 mesenchymal stem cells per square centimetre embedded in an alginate matrix and, a sterile gauze, and (ii) surgical aids comprising cotton, wrap, and a wound cleaning agent; wherein at least about 90% of the mesenchymal stem cells remain viable when the pouch or sachet is stored at about 25° C. for a period of at least 6 months, or at least 12 months.

The present invention is further illustrated by the following example which is provided merely to be exemplary of the invention and do not limit the scope of the invention. Certain modifications and equivalents will be apparent to those skilled in the art and are intended to be included within the scope of the present invention.

### EXAMPLES

### Example 1: Topical Bandage/Patch Composition of Mesenchymal Stem Cells

Mesenchymal stem cells derived from adipose tissue of a subject were washed with 0.01M PBS and transferred to the bottom of disposable culture flasks pre-wetted with 10% FBS complete media (DMEM medium supplemented with 10% FBS), and incubated at 37° C. and 5% CO₂ in a humidified atmosphere for 1-2 hours. Media were changed every three days. Cells were trypsinized with 0.25% (w/v) trypsin and 0.02% (w/v) EDTA when they reached>80% confluence and sub-cultured at a density of 2×10⁴ cells/cm².

A 5 mL suspension containing mesenchymal stem cells (about 100×10⁸ cells) and 2% sodium alginate was prepared in a DMEM medium.

Procedure for preparation of a topical dressing composition in the form of bandage/patch:
a) sterile gauze sheet was placed on to stainless steel casting mould;
b) mesenchymal stem cells and alginate matrix were applied on the sterile gauze sheet;
c) the casting mould was placed in a bath of calcium chloride solution;
d) the casting mould was transferred to a bath containing phosphate buffer solution to wash excess of calcium;
e) gauze sheet was placed to a culture media flask for incubation;
f) the gauze sheet was incubated at 37° C. at 5% CO2 for 14 days; and
g) the incubated gauze sheet was placed in trilaminated pouch to obtain the topical dressing composition in the form of bandage/patch.

### Example 1A: Application of Topical Bandage/Patch Composition of Example 1 for Wound Healing

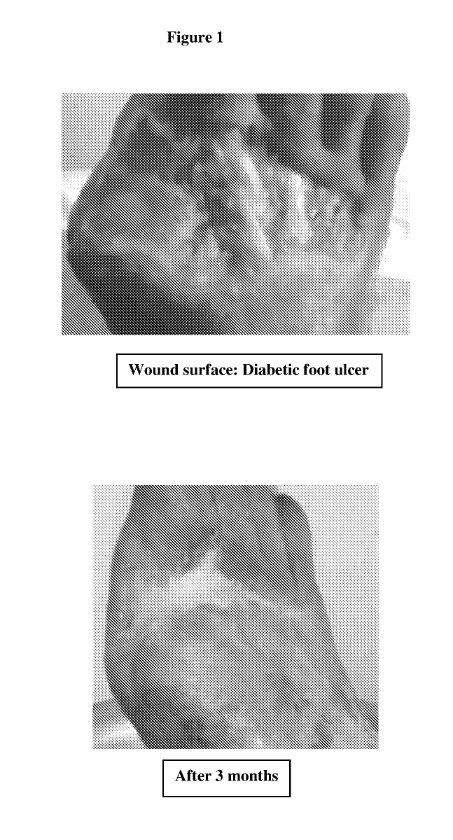

The topical bandage/patch has been used for the treatment of damaged skin tissue i.e. wound, in a subject. Application of topical dressing composition of Example 1 is suitable for autologous transfer or allogeneic transfer. According to Example 1, a topical bandage/patch has been prepared under sterile conditions. Before applying the bandage/patch, wound was flushed gently with sterile normal saline to remove the debris. The topical patch containing mesenchymal stem cells was applied on to the wounded skin surface. The topical bandage/patch was left on the wound for the period of 3 to 7 days. The topical bandage/patch application has been repeated at least 3 times and till the wound closure is observed. It is evident from the results from the Table 1, Table 2 and FIG. 1 that the wounded area has been repaired significantly and complete closure of wound as observed at the end of treatment of 3 months.

TABLE 1

| | Initial | | | |
|---|---|---|---|---|
| | (before | After application of topical patch | | |
| | treatment) | 1 month | 2 months | 3 months |
| Wound | 2232 | 1802 | 913 | 126 |
| surface area | | | | |
| in mm² | | | | |
| % Reduction | — | 19.26 | 59.09 | 94.35 |
| in wound | | | | |
| surface area | | | | |

TABLE 2

| | Initial | | | |
|---|---|---|---|---|
| | (before | After application of topical patch | | |
| | treatment) | 8 days | 15 days | 30 days |
| Wound | 1065 | 965 | 612 | 24 |
| surface area | | | | |
| in mm² | | | | |
| % Reduction | — | 9.38 | 42.53 | 97.74 |
| in wound | | | | |
| surface area | | | | |

### Example 1B: Stability Data of Mesenchymal Stem Cells for their Viability

The mesenchymal stem cells isolated and cultured are evaluated for their ability to differentiate using Adipogenic, Chondrogenic, and Osteogenic differentiation media. The cell surface markers are evaluated by Flow cytometry. The viability of cells is assayed by dye exclusion 7AAD analysis on the flow cytometer.

For study, six topical dressing compositions mentioned in table 3 (C1, C2, C3, C4, C5 and C6) were prepared as per procedure given in example 1.

TABLE 3

| | Duration | | Mesenchymal | Thickness of | % Viability at various | | | |
|---|---|---|---|---|---|---|---|---|
| | in | Storage | stem cells | topical dressing | Stability time point | | | |
| | culture | temperature | count per | (bandage/patch) | (in months) | | | |
| Compositions | (in weeks) | (in ° C.) | centimetre | in mm | Initial | 3 | 6 | 12 |
| C1 | 2 | 2-8 | 10,000 | 0.5 | 94.38 | 93.75 | 96.9 | 97.12 |
| C2 | 1 | 2-8 | 15,000 | 1.5 | 96.28 | 99.8 | 98.18 | 98.58 |
| C3 | 3 | 25 | 20,000 | 0.8 | 92.74 | 98.1 | 92.16 | 95.32 |
| C4 | 3 | 25 | 25,000 | 1.0 | 92.74 | 97.86 | 99.59 | 99.62 |
| C5 | 2 | 25 | 30,000 | 2.0 | 95.74 | 99.28 | 96.08 | 95.23 |
| C6 | 2 | 2-8 | 40,000 | 1.8 | 95.74 | 99.27 | 99.21 | 98.34 |

It is evident from the table 2 that at least about 80% of the mesenchymal stem cells remain viable when the composition is stored at about 4° C. and 25° C. for a period of 12 months.

### Example 2: A Topical Composition of Mesenchymal Stem Cells and Lyophilised Alginate Powder

Mesenchymal cells derived from adipose tissue of a subject were washed with 0.01M PBS and transferred to the bottom of disposable culture flasks pre-wetted with 10% FBS complete media (DMEM medium supplemented with 10% FBS), and incubated at 37° C. and 5% CO2 in a humidified atmosphere for 1-2 hours. Media were changed every three days. Cells were trypsinized with 0.25% (w/v) trypsin and 0.02% (w/v) EDTA when they reached>80% confluence and sub-cultured at a density of 2×10⁴ cells/cm².

A 5 mL suspension containing mesenchymal stem cells (about 100×10⁸ cells) was prepared in a DMEM medium. The 2% w/v alginate polymerised gel is prepared and lyophilised.

### Example 2A: Application of a Topical Dressing Composition of Example 2 for Wound Healing

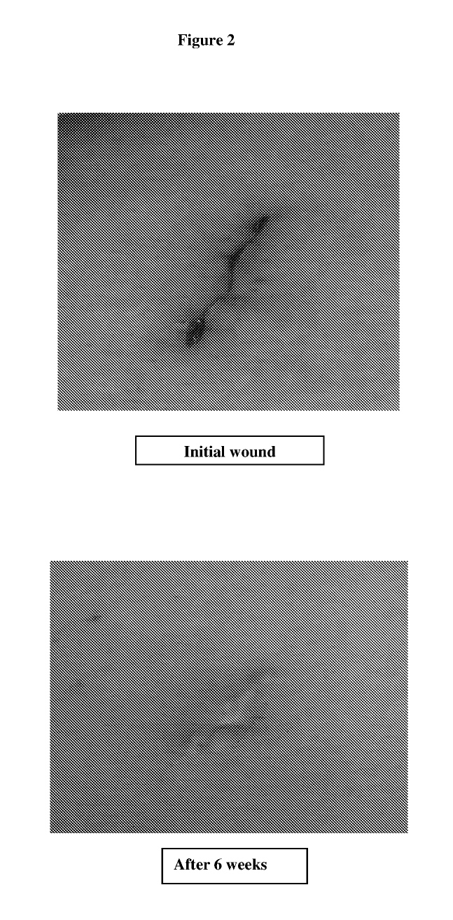

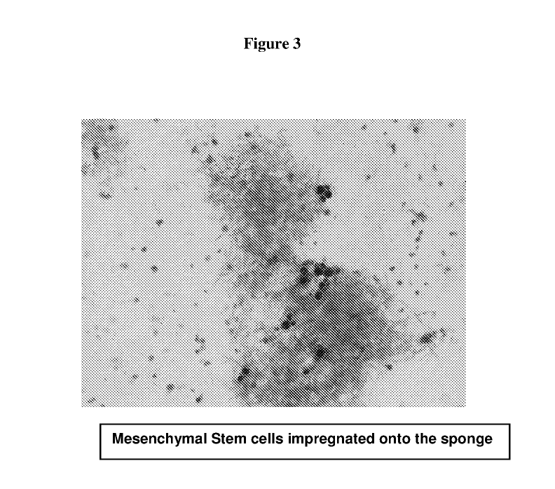

The topical dressing composition has been used for the treatment of damaged skin tissue i.e. wound, in a subject. Application of topical dressing composition of Example 2 is suitable for autologous transfer or allogeneic transfer. According to Example 2, the composition has been prepared under aseptic conditions. Before applying the composition, wound was flushed gently with sterile normal saline to remove the debris. The composition containing mesenchymal stem cells was applied on to the wounded skin surface. The lyophilised powder was applied on to application of mesenchymal stem cells; thereby formed a sponge on to the wound surface. The mesenchymal stem cells were impregnated on to the sponge as shown in FIG. 3. The composition was left on the wound surface for the period of 1 to 3 days. The composition application has been repeated at least 3 times and till the wound closure was observed. It is evident from the results from the Table 4 and FIG. 2 that the wound area was repaired significantly and closure of wound as observed at the end of treatment of 6 weeks.

TABLE 4

| | | After application of | | |
|---|---|---|---|---|
| | Initial | topical composition | | |
| | (before treatment) | 2 weeks | 4 weeks | 6 weeks |
| Wound surface area | 1224 | 832 | 482 | 92 |
| in mm² | | | | |
| % Reduction in | — | 24.83 | 54.11 | 95.35 |
| wound surface area | | | | |

### Example 3: A Dual Chambered Spray Composition of Mesenchymal Stem Cells, Alginate and Calcifying Agent

Mesenchymal cells derived from adipose tissue of a subject were washed with 0.01M PBS and transferred to the bottom of disposable culture flasks pre-wetted with 10% FBS complete media (DMEM medium supplemented with 10% FBS), and incubated at 37° C. and 5% CO2 in a humidified atmosphere for 1-2 hours. Media were changed every three days. Cells were trypsinized with 0.25% (w/v) trypsin and 0.02% (w/v) EDTA when they reached>80% confluence and sub-cultured at a density of 2×10⁴ cells/cm².

A 5 mL suspension containing mesenchymal stem cells and 2% sodium alginate was prepared in HypoThermosol® medium. Separately a 5 mL solution of CaCl₂ (150 mM) was prepared. The mesenchymal stem cells-alginate suspension and calcium chloride solution was filled aseptically into container with two separate chambers and a valve to obtain the dual chambered spray as below:

### Part A: Mesenchymal Stem Cell Component (First Chamber)

| Ingredient | Quantity (5 ml) |
|---|---|
| Mesenchymal stem cell component | 40,000,000* mesenchymal stem cells |
| | per mL. |
| Sodium Alginate | 2% by weight of composition |
| HypoThermosol ® media | q.s. |

Note:
*mesenchymal stem cells amount may be selected from 40,000 to 80,000,000 cells per mL.

### Part B: Calcifying Component (Second Chamber)

| Ingredient | Quantity (5 ml) |
|---|---|
| Calcium Chloride solution | 150 mM |

### Example 3A: Application of a Topical Dressing Composition of Example 3 for Wound Healing

The topical dressing composition has been used for the treatment of damaged skin tissue i.e. wound, in a subject. Application of topical dressing composition of Example 3 is suitable for autologous transfer or allogeneic transfer. According to Example 3, the composition has been prepared under aseptic conditions. Before applying the composition, wound was flushed gently with sterile normal saline to remove the debris. The topical spray containing mesenchymal cells was applied on to the wounded skin surface. The composition was left on the wound surface for the period of 1 to 3 days. The composition application has been repeated at least 3 times and till the wound closure was observed. It is evident from the results from the Table 5 that the wound area was repaired significantly and closure of wound as observed at the end of treatment of 8 weeks.

TABLE 5

| | Initial | | | |
|---|---|---|---|---|
| | (before | After application of topical spray | | |
| | treatment) | 2 weeks | 4 weeks | 8 weeks |
| Wound surface area in mm² | 976 | 712 | 335 | 41 |
| % Reduction in wound | — | 27.04 | 65.67 | 95.79 |
| surface area | | | | |

### Example 4: A Gel Composition of Mesenchymal Stem Cells Embedded in an Alginate Matrix

Mesenchymal cells derived from adipose tissue of a subject were washed with 0.01M PBS and transferred to the bottom of disposable culture flasks pre-wetted with 10% FBS complete media (DMEM medium supplemented with 10% FBS), and incubated at 37° C. and 5% CO2 in a humidified atmosphere for 1-2 hours. Media were changed every three days. Cells were trypsinized with 0.25% (w/v) trypsin and 0.02% (w/v) EDTA when they reached>80% confluence and sub-cultured at a density of 2×10⁴ cells/cm².

A suspension containing mesenchymal stem cells and 2% sodium alginate was prepared in HypoThermosol® medium. A mixture of propylene glycol and hydroxy ethyl cellulose was prepared. The suspension containing mesenchymal stem cells was slowly added with mixing into the mixture of propylene glycol and hydroxy ethyl cellulose. Finally the volume was made with addition of saline (sodium chloride solution 0.9% w/v) to obtain a gel composition. All steps were performed under aseptic condition:

| | Gel compositions | | | |
|---|---|---|---|---|
| | Quantity (% by weight) | | | |
| Ingredient | A | B | C | D |
| Mesenchymal stem cell* | 70 | 60 | 50 | 80 |
| component in | | | | |
| HypoThermosol ® | | | | |
| media | | | | |
| Sodium alginate | 2% by weight of composition | | | |
| Propylene Glycol | 10 | 20 | 25 | 10 |
| Hydroxy ethyl cellulose | 15 | 10 | 20 | 5.0 |
| NaCl (0.9% w/v) | q.s. (up to | q.s. (up to | q.s. (up to | q.s. (up to |
| | 10 ml) | 10 ml) | 10 ml) | 10 ml) |

Note:
*mesenchymal stem cells amount contains 4,00,000 cells per mL. Mesenchymal stem cells amount may be selected from 40,000 to 80,000,000 cells per mL.

### Example 4A: Application of Topical Composition of Example 4 for Wound Healing

The topical composition has been used to repair skin wound of patients. According to Example 4, a topical composition has been prepared under aseptic conditions. Before applying the composition, wound was flushed gently with sterile normal saline to remove the debris. The topical composition containing mesenchymal cells was applied on to the wounded skin surface. The topical composition was left on the wound for the period of 1 to 3 days. The topical composition application has been repeated at least 3 times and till the wound closure was observed. It is evident from Table 6 that the wound area was repaired significantly and closure of wound as observed at the end of treatment of 6 weeks.

TABLE 6

| | | After application of | | |
|---|---|---|---|---|
| | Initial | topical composition | | |
| | (before treatment) | 2 weeks | 4 weeks | 6 weeks |
| Wound surface area | 1334 | 952 | 532 | 102 |
| in mm² | | | | |
| % Reduction in | — | 28.63 | 60.11 | 92.35 |
| wound surface area | | | | |

### Example 5: A Topical Dressing Composition of Mesenchymal Stem Cells

Mesenchymal cells derived from adipose tissue of a subject were washed with 0.01M PBS (Phosphate buffered saline) and transferred to the bottom of disposable culture flasks pre-wetted with 10% FBS (Fetal Bovine Serum) complete media (DMEM (Dulbecco's Modified Eagle's medium) medium supplemented with 10% FBS), and incubated at 37° C. and 5% CO₂ in a humidified atmosphere for 1-2 hours. Media were changed every three days. Cells were trypsinized with 0.25% (w/v) trypsin and 0.02% (w/v) EDTA when they reached>80% confluence and sub-cultured at a density of 2×10⁴ cells/cm². A 5 mL suspension containing mesenchymal stem cells and 2% sodium alginate was prepared in HypoThermosol® medium to obtain a topical composition. All steps have been performed under aseptic condition as below:

| Ingredient | Quantity (5 ml) |
|---|---|
| Mesenchymal stem cell component | 40,000,000* mesenchymal stem cells |
| with 2% sodium alginate | per mL. |
| HypoThermosol ® media | q.s. |

Note:
*mesenchymal stem cells amount may be selected from 40,000 to 80,000,000 cells per mL.

### Example 6: A Topical Dressing Composition of Mesenchymal Stem Cells

Mesenchymal cells derived from adipose tissue of a subject were washed with 0.01M PBS (Phosphate buffered saline) and transferred to the bottom of disposable culture flasks pre-wetted with 10% FBS (Fetal Bovine Serum) complete media (DMEM (Dulbecco's Modified Eagle's medium) medium supplemented with 10% FBS), and incubated at 37° C. and 5% CO₂ in a humidified atmosphere for 1-2 hours. Media were changed every three days. Cells were trypsinized with 0.25% (w/v) trypsin and 0.02% (w/v) EDTA when they reached>80% confluence and sub-cultured at a density of 2×10⁴ cells/cm².

A 5 mL suspension containing mesenchymal stem cells was prepared in HypoThermosol® medium to obtain a topical composition. All steps have been performed under aseptic condition as below:

| Ingredient | Example 6A | Example 6B |
|---|---|---|
| Mesenchymal stem cell | 40,000,000* | 40,000,000* |
| component | mesenchymal | mesenchymal |
| | stem cells per mL. | stem cells per mL. |
| Chitosan solution | 2% | — |
| (as topical matrix) | | |
| Hyaluronic acid solution | — | 2% |
| (as topical matrix) | | |

Note:
*mesenchymal stem cells amount may be selected from 40,000 to 80,000,000 cells per mL.

1. A storage-stable topical dressing composition in the form of a bandage or patch for the treatment of damaged skin tissue in a subject, wherein said bandage or patch has thickness in the range of about 0.5 mm to about 10 mm, and comprises about 10,000 to about 40,000 mesenchymal stem cells per square centimetre embedded in a topical alginate matrix and optionally, a pharmaceutically acceptable excipient, wherein at least about 80% of the contained mesenchymal stem cells remain viable when the composition is stored at about 25° C. for a period of at least 6 months.

2. The composition of claim 1, wherein the damaged skin tissue includes traumatic wound, surgical wound, diabetic ulcer, pressure ulcer, venous ulcer, a scar, burn, a skin lesion, eczema, and a skin ulcer.

3. The composition of claim 1, wherein at least about 85% of the contained mesenchymal stem cells remain viable when the composition is stored at about 25° C. for a period of at least about 12 months.

4. The composition of claim 1, wherein the topical dressing comprises about 10,000 to about 35,000 mesenchymal stem cells per square centimetre.

5. The composition of claim 1, wherein the topical dressing comprises about 10,000 to about 30,000 mesenchymal stem cells per square centimetre.

6. The composition of claim 1, wherein the topical alginate matrix comprises 2% w/v of alginate solution.

7. (canceled)

8. The composition of claim 1, wherein the bandage or patch has thickness in the range of about 1 mm to about 5 mm.

9. The pharmaceutical composition of claim 1, wherein the mesenchymal stem cells are derived from adipose tissue, bone marrow, Whartons jelly dental tissue or umbilical cord of a subject.

10. The composition of claim 1, wherein the mesenchymal stem cells are suitable for autologous transfer or allogeneic transfer.

11. A storage-stable topical dressing composition in the form of a bandage or patch for the treatment of damaged skin tissue in a subject, wherein said bandage or patch has thickness in the range of about 1 mm to about 5 mm, and comprises about 10,000 to about 40,000 mesenchymal stem cells per square centimetre embedded in an topical matrix comprising 2% alginate solution and, optionally a pharmaceutically acceptable excipient, wherein at least about 85% of the mesenchymal stem cells remain viable when the composition is stored at about 25° C. for a period of at least 12 months.

12. (canceled)

13. (canceled)

14. (canceled)

15. The composition of claim 10, wherein the mesenchymal stem cells are derived from adipose tissue.

16. (canceled)

17. The method of preparation of a storage stable topical dressing composition of claim 10, wherein said method comprises the steps of:
a) placing sterile gauze sheet on to stainless steel casting mould;
b) applying mesenchymal stem cells and alginate matrix on the sterile gauze sheet;
c) placing the casting mould in a bath of calcium chloride solution;
d) transferring of the casting mould to a bath containing phosphate buffer solution to wash excess of calcium;
e) placing of gauze sheet to a culture media flask for incubation;
f) incubating the gauze sheet at 37° C. at 5% CO₂ for 14 days; and
g) placing the incubated gauze sheet in trilaminated pouch to obtain the topical dressing composition.

18. A storage-stable topical dressing composition in the form of a dual chambered spray for the treatment of damaged skin tissue in a subject, said composition comprises (i) mesenchymal stem cells and topical matrix component in one chamber, and (ii) gelling component in another chamber, wherein more than about 80% of the mesenchymal stem cells remain viable when the composition is stored at about 25° C. for a period of at least 6 months.

19. A storage-stable topical dressing composition in the form of a gel for the treatment of damaged skin tissue in a subject, wherein said gel comprises mesenchymal stem cells embedded in a topical matrix comprising 2% w/v of alginate solution and, optionally a pharmaceutically acceptable excipient, wherein more than about 80% of the mesenchymal stem cells remain viable when the topical gel is stored at about 25° C. for a period of at least 6 months.

20. A method of treatment of damaged skin tissue in a subject, said method comprising: (i) applying to the damaged skin tissue area a composition containing about 10,000 to about 40,000 mesenchymal stem cells per square centimetre and a pharmaceutically acceptable excipient, and (ii) applying a topical matrix comprising 2% w/v of alginate solution in the form of a lyophilized powder and optionally a pharmaceutically acceptable excipient onto the mesenchymal stem cell composition, thereby forming an in situ sponge on the damaged skin tissue.
